(19) United States
(12) Patent Application Publication
(10) Pub. No.: US 2024/0293006 A1
(43) Pub. Date: Sep. 5, 2024

(54) ENDOSCOPE DISTAL END AND ENDOSCOPE

(71) Applicant: OLYMPUS MEDICAL SYSTEMS CORP., Tokyo (JP)

(72) Inventor: Takahiro SUWA, Tokyo (JP)

(73) Assignee: OLYMPUS MEDICAL SYSTEMS CORP., Tokyo (JP)

(21) Appl. No.: 18/589,859

(22) Filed: Feb. 28, 2024

Related U.S. Application Data
(60) Provisional application No. 63/449,350, filed on Mar. 2, 2023.

(51) Int. Cl. A61B 1/00; A61B 1/018

(52) U.S. Cl. CPC A61B 1/00098; A61B 1/018

(57) ABSTRACT

A distal end portion for use with an endoscope includes: a distal end body having an insertion hole, the insertion hole configured to accept a treatment tool inserted therethrough; a shaft rotatably disposed relative to the distal end body; and a raising base configured to rotate with the shaft, and be movable between a raised state where the raising base has been raised relative to a longitudinal direction of the distal end body and a laid state where the raising base has been laid relative to the longitudinal direction, and the raising base comprising; a shaft hole having an interior configured to be engaged with an exterior of the shaft; and a treatment tool guiding surface configured to abut against the treatment tool to adjust a direction in which the treatment tool protrudes.

### CROSS-REFERENCE TO RELATED APPLICATION

This application is based on and claims priority under 35 U.S.C. § 119 to U.S. Provisional Application No. 63/449,350, filed Mar. 2, 2023, the entire contents of which are incorporated herein by reference.

### BACKGROUND

The present disclosure relates to endoscope distal ends and endoscopes.

An endoscope for observation of the interior of a subject, such as a human, has been known conventionally, the endoscope including a flexible and elongated insertion portion to be inserted into the subject (see, for example, International Publication Pamphlet No. WO 2016/021231). An endoscope described in International Publication Pamphlet No. WO 2016/021231 has a distal end component, a rotation mechanisms, and a treatment tool raising stand that are described below and provided at a distal end of an insertion portion thereof.

The distal end component has a treatment tool insertion hole where a treatment tool, such as a puncture needle, is inserted through.

The rotation mechanism includes a shaft portion that is arranged to be rotatable with respect to the distal end component.

The treatment tool raising stand is set, by rotating with the shaft portion, in a raised state where the treatment tool raising stand has been raised or a laid state where the treatment tool raising stand has been laid, with respect to the longitudinal direction of the distal end component, and adjusts, by abutting against the treatment tool that has protruded from the treatment tool insertion hole, the direction in which the treatment tool protrudes. This treatment tool raising stand has, provided therein, a hole to be engaged with the shaft portion and a treatment tool guiding surface that adjusts, by abutting against the treatment tool, the direction in which the treatment tool protrudes.

### SUMMARY

In some embodiments, a distal end portion for use with an endoscope includes: a distal end body having an insertion hole, the insertion hole configured to accept a treatment tool inserted therethrough; a shaft rotatably disposed relative to the distal end body; and a raising base configured to rotate with the shaft, and be movable between a raised state where the raising base has been raised relative to a longitudinal direction of the distal end body and a laid state where the raising base has been laid relative to the longitudinal direction, and the raising base comprising; a shaft hole having an interior configured to be engaged with an exterior of the shaft; and a treatment tool guiding surface configured to abut against the treatment tool to adjust a direction in which the treatment tool protrudes, the treatment tool guiding surface provided on at least a proximal end side of the raising base. The interior of the shaft hole comprises a first surface configured to abut against a corresponding portion of the exterior of the shaft, the first surface is positioned at a shortest distance from the treatment tool guiding surface relative to other portions of the interior of the shaft hole, and the treatment tool guiding surface is configured to intersect the longitudinal direction in the laid state.

In some embodiments, an endoscope includes: an insertion portion having an distal end portion at a distal end of the insertion portion, the insertion portion being configured to be inserted into a subject. The distal end portion includes: a distal end body having an insertion hole, the insertion hole configured to accept a treatment tool inserted therethrough; a shaft rotatably disposed relative to the distal end body; and a raising base configured to rotate with the shaft, and be movable between a raised state where the raising base has been raised relative to a longitudinal direction of the distal end body and a laid state where the raising base has been laid relative to the longitudinal direction, and the raising base comprising; a shaft hole having an interior configured to be engaged with an exterior of the shaft; and a treatment tool guiding surface configured to abut against the treatment tool to adjust a direction in which the treatment tool protrudes, the treatment tool guiding surface provided on at least a proximal end side of the raising base, wherein the interior of the shaft hole comprises a first surface configured to abut against a corresponding portion of the exterior of the shaft, the first surface is positioned at a shortest distance from the treatment tool guiding surface relative to other portions of the interior of the shaft hole, and the treatment tool guiding surface is configured to intersect the longitudinal direction in the laid state.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to an embodiment.

FIG. 2 is a diagram illustrating a configuration of an endoscope distal end.

FIG. 3 is a diagram illustrating the configuration of the endoscope distal end.

FIG. 4 is a diagram illustrating a configuration of a treatment tool raising stand.

FIG. 5 is a diagram illustrating the configuration of the treatment tool raising stand.

FIG. 6 is a diagram for description of a first modified example of the embodiment.

FIG. 7 is a diagram for description of a second modified example of the embodiment.

### DETAILED DESCRIPTION

Modes for implementing the disclosure (hereinafter, “embodiments”) will hereinafter be described by reference to the drawings. The disclosure is not limited by the embodiments described below. Furthermore, the same reference sign is assigned to portions that are the same, throughout the drawings.

### Schematic Configuration of Endoscope System

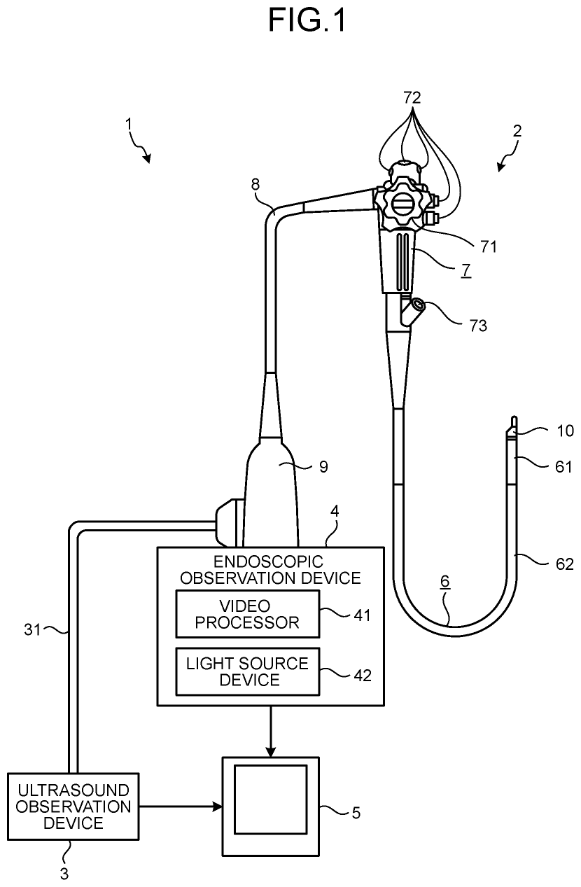

FIG. 1 is a diagram illustrating an endoscope system 1 according to an embodiment.

The endoscope system 1 is a system for ultrasound diagnosis of the interior of a subject, such as a human, by use of an ultrasound endoscope. This endoscope system 1 includes, as illustrated in FIG. 1, an ultrasound endoscope 2, an ultrasound observation device 3, an endoscopic observation device 4, and a display device 5.

The ultrasound endoscope 2 corresponds to an endoscope. Part of this ultrasound endoscope 2 is capable of being inserted into a subject, and the ultrasound endoscope 2 has a function of transmitting ultrasound pulses to a body wall inside the subject, receiving ultrasound echoes reflected by the subject, and outputting echo signals, and a function of outputting an image signal by capturing an image of the interior of the subject. A detailed configuration of the ultrasound endoscope 2 will be described in a later section, “Configuration of Ultrasound Endoscope”.

In this embodiment, the ultrasound endoscope 2 is adopted as the endoscope, but without being limited to the ultrasound endoscope 2, an endoscope, such as a duodenoscope, not having an ultrasound probe 11, may be adopted instead.

The ultrasound observation device 3 is electrically connected to the ultrasound endoscope 2 via an ultrasound cable 31 (FIG. 1), and outputs a pulse signal to the ultrasound endoscope 2 and inputs an echo signal from the ultrasound endoscope 2, via the ultrasound cable 31. The ultrasound observation device 3 generates an ultrasound image by performing predetermined processing of the echo signal.

A later described endoscope connector 9 (FIG. 1) of the ultrasound endoscope 2 is detachably connected to the endoscopic observation device 4. The endoscopic observation device 4 includes, as illustrated in FIG. 1, a video processor 41 and a light source device 42.

The video processor 41 receives an image signal from the ultrasound endoscope 2 via the endoscope connector 9. The video processor 41 generates an endoscopic image by performing predetermined processing of the image signal. The light source device 42 supplies illumination light for illuminating the interior of a subject, to the ultrasound endoscope 2 via the endoscope connector 9.

The display device 5 is a display using liquid crystal or organic electro luminescence (EL), and displays thereon, for example, an ultrasound image generated by the ultrasound observation device 3 or an endoscopic image generated by the endoscopic observation device 4.

### Configuration of Ultrasound Endoscope

The ultrasound endoscope 2 includes, as illustrated in FIG. 1, an insertion portion 6, an operating unit 7, a universal cord 8, and the endoscope connector 9.

“Distal” referred to hereinafter means being near, toward, or in the direction of a distal end of the insertion portion 6 (forward in the direction of insertion into a subject). Furthermore, “proximal” referred to hereinafter means being near, toward, or in the direction of an end away from the distal end of the insertion portion 6.

The insertion portion 6 is a portion to be inserted into a subject. This insertion portion 6 includes, as illustrated in FIG. 1, an endoscope distal end 10, a bending portion 61, and a flexible tube 62.

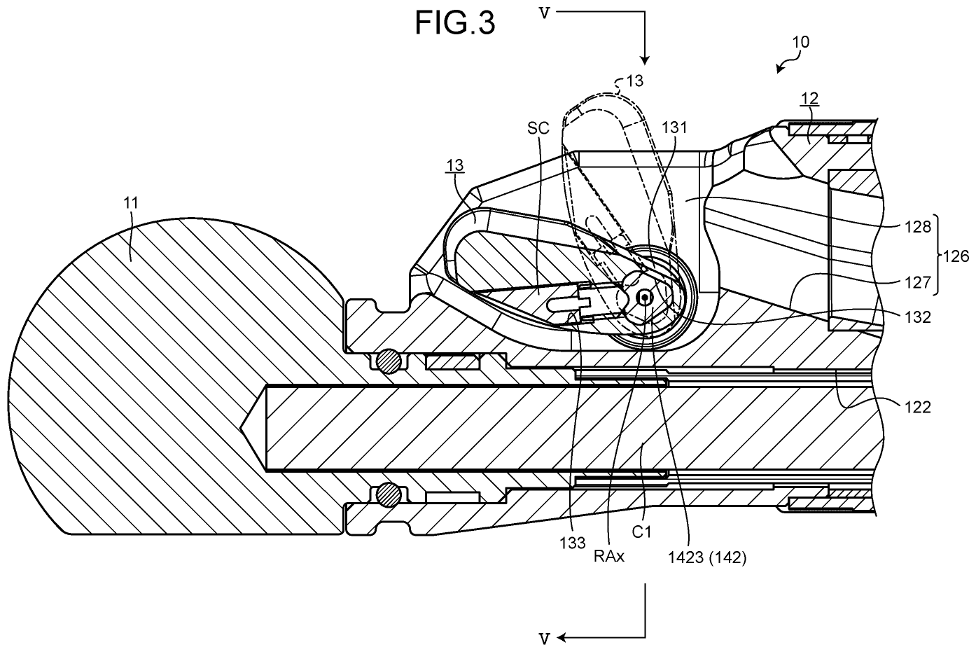

Inside the insertion portion 6, the operating unit 7, the universal cord 8, and the endoscope connector 9: a light guide (not illustrated in the drawings) that transmits illumination light supplied from the light source device 42, a transducer cable C1 (see FIG. 3) that transmits the above described pulse signal and echo signal, and a signal cable (not illustrated in the drawings) that transmits an image signal have been laid; and a duct line (not illustrated in the drawings) for circulating fluid has also been provided.

The endoscope distal end 10 (distal end portion) is provided at the distal end of the insertion portion 6.

A detailed configuration of the endoscope distal end 10 will be described in a later section, “Configuration of Endoscope Distal End”.

The bending portion 61 is connected near a proximal end of the endoscope distal end 10 and is bendable.

The flexible tube 62 is connected near a proximal end of the bending portion 61 and has flexibility.

The operating unit 7 is a portion that is connected near a proximal end of the insertion portion 6 and that receives various operations from, for example, a medical doctor. This operating unit 7 includes, as illustrated in FIG. 1, a bending knob 71 for operating the bending portion 61 to bend, and plural operating portions 72 for performing various operations.

Furthermore, the operating unit 7 has, provided therein, a treatment tool insertion port 73 connected to a tube (not illustrated in the drawings) provided inside the bending portion 61 and the flexible tube 62, the treatment tool insertion port 73 being for insertion of a treatment tool (not illustrated in the drawings) into the tube.

The universal cord 8 is a cord that extends from the operating unit 7 and that has, arranged therein: the above mentioned light guide (not illustrated in the drawings) and transducer cable C1; the above described signal cable (not illustrated in the drawings); and the above mentioned duct line (not illustrated in the drawings).

The endoscope connector 9 is provided at an end portion of the universal cord 8. The ultrasound cable 31 is connected to the endoscope connector 9 and the endoscope connector 9 is connected to the video processor 41 and the light source device 42 by being plugged into the endoscopic observation device 4.

### Configuration of Endoscope Distal End

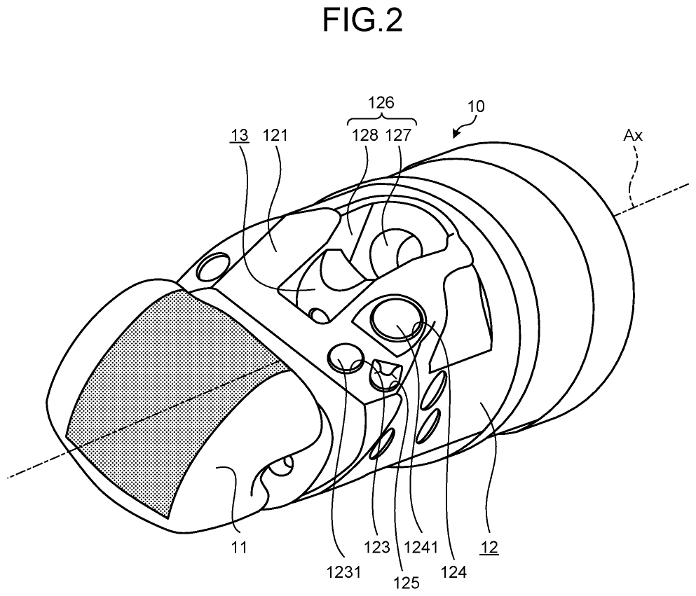

FIG. 2 and FIG. 3 are diagrams illustrating a configuration of the endoscope distal end 10. Specifically, FIG. 2 is a perspective view illustrating the endoscope distal end 10. FIG. 3 is a diagram illustrating a cross section of the endoscope distal end 10, the cross section having been cut along a plane including a central axis Ax (FIG. 2) of the insertion portion 6.

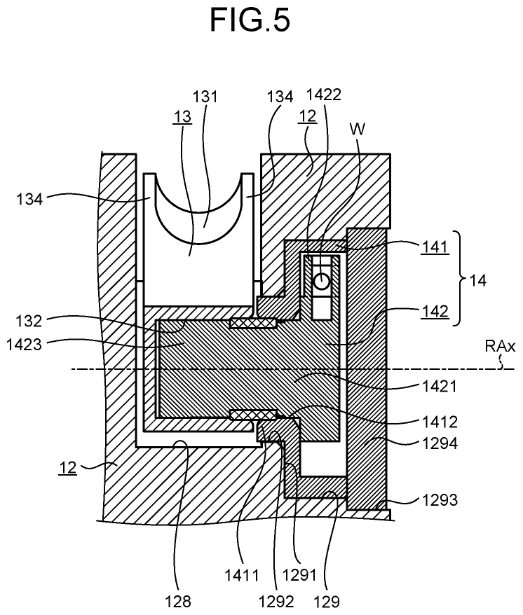

The endoscope distal end 10 includes, as illustrated in FIG. 2 and FIG. 3, an ultrasound probe 11, a distal end component 12, a treatment tool raising stand 13, and a rotation mechanism 14 (see FIG. 5).

The ultrasound probe 11 is a convex ultrasound probe and has plural ultrasound transducers (not illustrated in the drawings) regularly arranged in a state of forming a convex circular arc. The ultrasound probe 11 is not necessarily a convex ultrasound probe and a radial ultrasound probe may be adopted instead.

Each of the ultrasound transducers includes an acoustic lens, a piezoelectric element, and a matching layer, and acquires ultrasound echoes contributing to an ultrasound tomographic image of the interior of a subject, the interior being more inside than a body wall of the subject.

The ultrasound probe 11 converts a pulse signal input from the ultrasound observation device 3 via the ultrasound cable 31 and the transducer cable C1 into ultrasound pulses and transmits the ultrasound pulses into a subject. Furthermore, the ultrasound probe 11 converts ultrasound echoes reflected inside the subject, into an electric echo signal, and outputs the electric echo signal via the transducer cable C1 and the ultrasound cable 31, to the ultrasound observation device 3.

The ultrasound probe 11 described above is arranged, as illustrated in FIG. 3, nearer to the distal end (lower in FIG. 3) than the treatment tool raising stand 13 is.

The distal end component 12 (distal end body) is a rigid portion made of, for example, a resin material, and has an approximately cylindrical shape extending along the central axis Ax.

This distal end component 12 has an inclined surface 121 (FIG. 2) provided on an outer peripheral surface near a distal end of the distal end component 12, the inclined surface 121 making the distal end component 12 tapered toward the distal end of the distal end component 12.

The distal end component 12 has, provided therein, for example: an attachment hole 122 (FIG. 3) penetrating the distal end component 12 from a proximal end to the distal end of the distal end component 12; and an illumination hole 123 (FIG. 2), an imaging hole 124 (FIG. 2), a gas and water feeding hole 125 (FIG. 2), and a treatment tool channel 126 (FIG. 2 and FIG. 3) that each penetrate the distal end component 12 from the proximal end to the inclined surface 121.

The attachment hole 122 is a hole where the ultrasound probe 11 is to be attached. The transducer cable C1 electrically connected to the ultrasound probe 11 is inserted inside the attachment hole 122, as illustrated in FIG. 3.

The illumination hole 123 has, arranged therein, an output end of the above mentioned light guide (not illustrated in the drawings) and an illumination lens 1231 (FIG. 2) that illuminates the interior of a subject with illumination light output from the output end of the light guide.

The imaging hole 124 has, arranged therein: an objective optical system 1241 (FIG. 2) that condenses light (subject image) that has been emitted into a subject and reflected in the subject; and an imaging element (not illustrated in the drawings) that captures the subject image condensed by the objective optical system 1241. An image signal captured by the imaging element is transmitted to the endoscopic observation device 4 (the video processor 41) via the above mentioned signal cable (not illustrated in the drawings).

The gas and water feeding hole 125 is part of the duct line (not illustrated in the drawings) mentioned above and is a hole for cleaning an outer surface of the objective optical system 1241 by allowing gas or water to be fed to the imaging hole 124.

The treatment tool channel 126 is a passage that lets a treatment tool (not illustrated in the drawings) protrude outside, the treatment tool having been inserted through the above mentioned tube (not illustrated in the drawings) inside the insertion portion 6 from the treatment tool insertion port 73. This treatment tool channel 126 includes, as illustrated in FIG. 3, a treatment tool insertion hole 127 (insertion hole) and a storage groove 128.

The treatment tool insertion hole 127 is a portion extending from the proximal end of the distal end component 12 toward the distal end of the distal end component 12, the portion being where a treatment tool (not illustrated in the drawings) is to be inserted through.

The storage groove 128 is a groove that is in communication with the treatment tool insertion hole 127 and that extends toward the distal end from the treatment tool insertion hole 127 along the central axis Ax.

The treatment tool raising stand 13 (raising base) is stored inside the storage groove 128, rotatably about a rotation axis RAx (FIG. 3). The treatment tool raising stand 13 is set, by rotating about the rotation axis RAx, in a raised state where the treatment tool raising stand 13 has been raised or a laid state where the treatment tool raising stand 13 has been laid, with respect to the central axis Ax, and the treatment tool raising stand 13 adjusts, by abutting against a treatment tool (not illustrated in the drawings) that has been inserted through the treatment tool insertion hole 127 into the storage groove 128, the direction in which the treatment tool protrudes. In FIG. 3, the treatment tool raising stand 13 that has been set in the raised state is illustrated with dashed and dotted lines and the treatment tool raising stand 13 that has been set in the laid state is illustrated with solid lines.

A detailed configuration of the treatment tool raising stand 13 will be described in a later section, “Configuration of Treatment Tool Raising Stand”.

The rotation mechanism 14 is a mechanism that rotates the treatment tool raising stand 13 according to operation of the operating unit 7 by, for example, a medical doctor.

A detailed configuration of the rotation mechanism 14 will be described in a later section, “Configuration of Treatment Tool Raising Stand”.

### Configuration of Treatment Tool Raising Stand

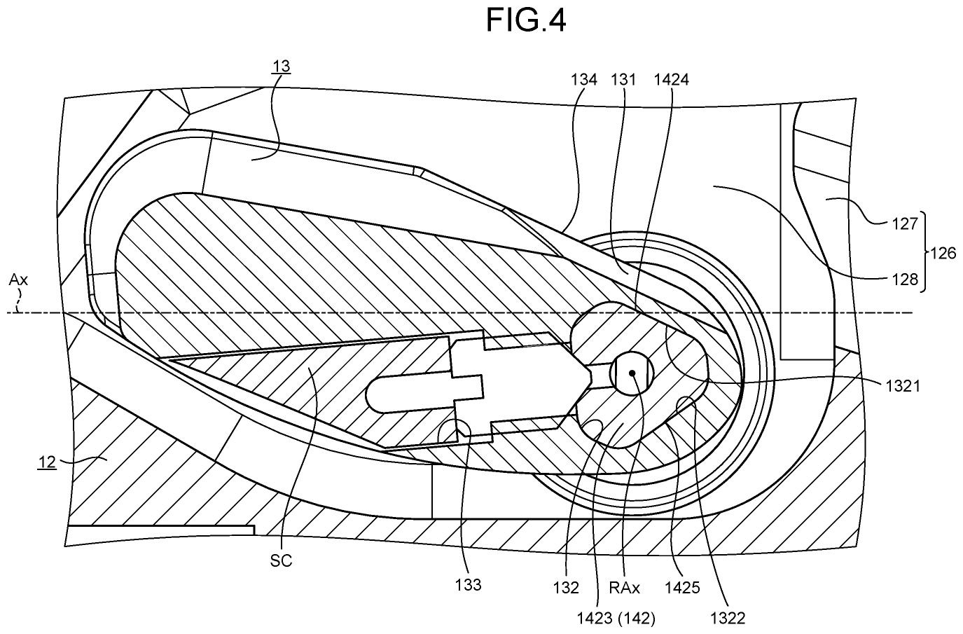

FIG. 4 and FIG. 5 are diagrams illustrating a configuration of the treatment tool raising stand 13. Specifically, FIG. 4 is an enlarged view of part of FIG. 3. FIG. 5 is a sectional view of the endoscope distal end 10 cut at a position of a line V-V in FIG. 3 along a plane orthogonal to the central axis Ax of the insertion portion 6.

A configuration of the rotation mechanism 14 will be described first, before description of the configuration of the treatment tool raising stand 13.

As illustrated in FIG. 5, the rotation mechanism 14 is arranged inside a recessed portion 129 that is positioned laterally to the storage groove 128 and that is recessed toward the storage groove 128 from an outer surface of the distal end component 12.

A bottom portion 1291 of the recessed portion 129 is formed of a side wall 1281 (FIG. 5) of the storage groove 128, the side wall 1281 being one of side walls forming the storage groove 128. The side wall 1281 is a wall orthogonal to the rotation axis RAx.

Furthermore, a first through hole 1292 that penetrates the side wall 1281 and lets the recessed portion 129 and the storage groove 128 communicate with each other is provided, as illustrated in FIG. 5, on the rotation axis RAx of the bottom portion 1291. This first through hole 1292 has a circular shape coaxial with the rotation axis RAx in a plan view.

Furthermore, a lid 1294 is attached to an opening 1293 of the recessed portion 129, as illustrated in FIG. 5. This lid 1294 has a planar shape that is approximately the same as that of the opening 1293 and closes the recessed portion 129 by being attached to the opening 1293.

The rotation mechanism 14 includes a bearing portion 141 and a shaft portion 142, as illustrated in FIG. 5.

As illustrated in FIG. 5, the bearing portion 141 has a bearing body 1411 having a second through hole 1412 and a ring shape and is installed inside the recessed portion 129 by means of a screw (not illustrated in the drawings) in a state where the bearing body 1411 has been inserted through the first through hole 1292. The bearing portion 141 supports the shaft portion 142 by means of the bearing body 1411 such that that shaft portion 142 is rotatable about the rotation axis RAx.

The shaft portion 142 includes, as illustrated in FIG. 5, a shaft portion body 1421 and an arm 1422.

The shaft portion body 1421 has an approximately cylindrical shape extending along the rotation axis RAx. This shaft portion body 1421 is supported rotatably about the rotation axis RAx in a state of having been inserted through the second through hole 1412. Furthermore, the shaft portion body 1421 has a protruding portion 1423 (FIG. 5) arranged to protrude in the storage groove 128. The protruding portion 1423 is connected to the treatment tool raising stand 13 arranged in the storage groove 128.

The arm 1422 is engaged with one end of a wire W that is fixed to an outer peripheral surface of the shaft portion body 1421 and that advances and retracts according to user operations performed by, for example, a medical doctor, on the operating unit 7.

When the wire W advances or retracts according to a user operation performed by, for example, a medical doctor, on the operating unit 7, the arm 1422 rotates integrally with the shaft portion body 1421 and the treatment tool raising stand 13 about the rotation axis RAx. That is, rotation of the treatment tool raising stand 13 adjusts the direction in which a treatment tool (not illustrated in the drawings) protrudes, the treatment tool having been inserted through the treatment tool insertion hole 127 into the storage groove 128.

The treatment tool raising stand 13 is a columnar body extending along the rotation axis RAx.

This treatment tool raising stand 13 has, provided therein, as illustrated in FIG. 4 and FIG. 5, a treatment tool guiding surface 131, a hole 132 (shaft hole), a screw hole 133, and a third plane 134.

The treatment tool guiding surface 131 is a surface that is provided leftward in FIG. 4 on an outer peripheral surface of the treatment tool raising stand 13 that is a columnar body, the outer peripheral surface being near a proximal end of the treatment tool raising stand 13 (upward in FIG. 4), and that adjusts, by abutting against a treatment tool (not illustrated in the drawings) that has been inserted through the treatment tool insertion hole 127 into the storage groove 128, the direction in which the treatment tool protrudes.

As illustrated in FIG. 5, a cross section of the treatment tool guiding surface 131 has a circular arc shape (curved shape) recessed downward in FIG. 5, the cross section being orthogonal to the central axis Ax. Furthermore, a cross section of the treatment tool guiding surface 131 has a linear shape, as illustrated in FIG. 4, the cross section being cut along a plane including the central axis Ax. When the treatment tool raising stand 13 has been set in the laid state, the treatment tool guiding surface 131 extends in a direction that is the same as the direction in which a distal end portion of the treatment tool insertion hole 127 extends (FIG. 3), with a proximal end (up in FIG. 3) of the treatment tool guiding surface 131 being positioned rightward in FIG. 3 and a distal end (down in FIG. 3) of the treatment tool guiding surface 131 being positioned leftward in FIG. 3. That is, the treatment tool guiding surface 131 intersects the central axis Ax when the treatment tool raising stand 13 has been set in the laid state.

The hole 132 is a blind hole that extends from one of side surfaces of the treatment tool raising stand 13 toward the other one of the side surfaces, as illustrated in FIG. 5, the one being near the recessed portion 129, the side surfaces both being along the rotation axis RAx, and the hole 132 is engaged with the protruding portion 1423 of the shaft portion 142. The hole 132 has an interior configured to be engaged with an exterior of the shaft portion 142.

This hole 132 has, as illustrated in FIG. 4, a first plane 1321 and a second plane 1322, provided therein.

The first plane 1321 is a plane that includes plural positions that are at the smallest distance between the first plane 1321 and the treatment tool guiding surface 131 and that abuts against an outer surface of the protruding portion 1423. Distances separating these plural positions from the treatment tool guiding surface 131 may all be the same or may differ from one another by about 0.2 mm to 0.5 mm.

The second plane 1322 is a plane that is positioned near a proximal end of the hole 132 (up in FIG. 4) and that abuts against the outer surface of the protruding portion 1423. An extended plane that is an extension of the first plane 1321 and an extended plane that is an extension of the second plane 1322 are at an acute angle to each other.

The outer surface of the protruding portion 1423 has, provided therein, a first plane 1424 that abuts against the first plane 1321 and a second plane 1425 that abuts against the second plane 1322.

As illustrated in FIG. 4, the screw hole 133 is a screw hole linearly penetrating the treatment tool raising stand 13 from a right side of an outer surface of the treatment tool raising stand 13 to an inner surface of the hole 132 excluding the first and second planes 1321 and 1322, the outer surface being near a distal end of the treatment tool raising stand 13 (down in FIG. 4). A screw SC is inserted into this screw hole 133. By being inserted into the screw hole 133, the screw SC pushes the protruding portion 1423 against the first and second planes 1321 and 1322.

As illustrated in FIG. 4 and FIG. 5, the third plane 134 is a plane provided on the outer surface of the treatment tool raising stand 13 and on both sides of the treatment tool guiding surface 131 along the central axis Ax.

The above described embodiment has the following effects.

In the endoscope distal end 10 according to the embodiment, when the treatment tool raising stand 13 has been set in the laid state, the treatment tool guiding surface 131 intersects the central axis Ax and extends in the same direction as the direction in which the distal end portion of the treatment tool insertion hole 127 extends. Furthermore, the hole 132 has the first plane 1321 that includes the plural positions that are at the smallest distance from the treatment tool guiding surface 131 and that abuts against the outer surface of the protruding portion 1423.

Therefore, with this positional relation between the treatment tool guiding surface 131 and the treatment tool insertion hole 127, the treatment tool guiding surface 131 will not bulge more than necessary.

Therefore, the endoscope distal end 10 according to the embodiment enables: prevention of a treatment tool being caught by the treatment tool guiding surface 131, the treatment tool having protruded from the treatment tool insertion hole 127; and improvement in insertability of the treatment tool.

Furthermore, the extended planes from the first and second planes 1321 and 1322 in the hole 132 are at an acute angle to each other. Therefore, this shape of the hole 132 prevents the treatment tool guiding surface 131 from bulging more than necessary and enables further improvement in the insertability of a treatment tool.

### Other Embodiments

Modes for implementing the disclosure have been described thus far, but the disclosure should not be limited only to the embodiment described above.

In the above described embodiment, the endoscope system 1 has both the function of generating an ultrasound image and the function of generating an endoscopic image, but without being limited to this embodiment, the endoscope system 1 may be configured to have only one of these functions.

In the above described embodiment, the endoscope system 1 may be an endoscope system for observation of the interior of a subject, such as a mechanical structure in the industrial field, without being limited to that in the medical field.

In the above described embodiment, a configuration of a first modified example or a second modified example described below may be adopted instead.

### First Modified Example

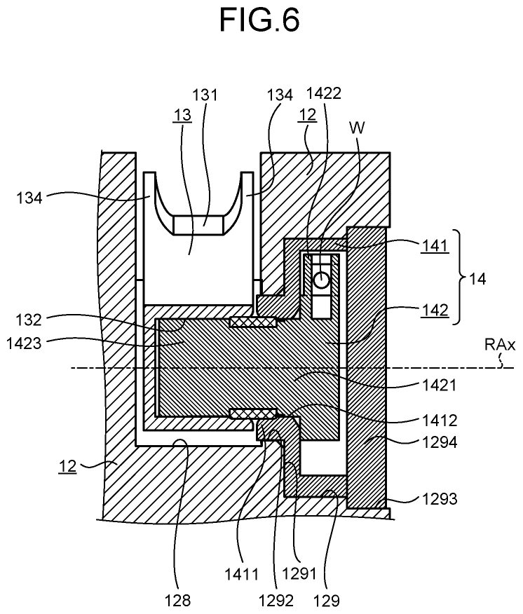

FIG. 6 is a diagram for description of a first modified example of the embodiment. Specifically, FIG. 6 a diagram corresponding to FIG. 5.

In the above described embodiment, the treatment tool guiding surface 131 may be shaped like a treatment tool guiding surface 131 of the first modified example illustrated in FIG. 6.

Specifically, a cross section of the treatment tool guiding surface 131 according to the first modified example has a linear shape parallel to a rotation axis RAx, as illustrated in FIG. 6, the cross section being orthogonal to a central axis Ax. Furthermore, a cross section of the treatment tool guiding surface 131 has a linear shape, the cross section being cut along a plane including the central axis Ax. When a treatment tool raising stand 13 has been set in a laid state, the treatment tool guiding surface 131 intersects the central axis Ax and extends in a direction that is the same as the direction in which a distal end portion of a treatment tool insertion hole 127 extends.

Even when the above described treatment tool guiding surface 131 according to the first modified example is adopted, effects similar to those of the embodiment described above are achieved.

### Second Modified Example

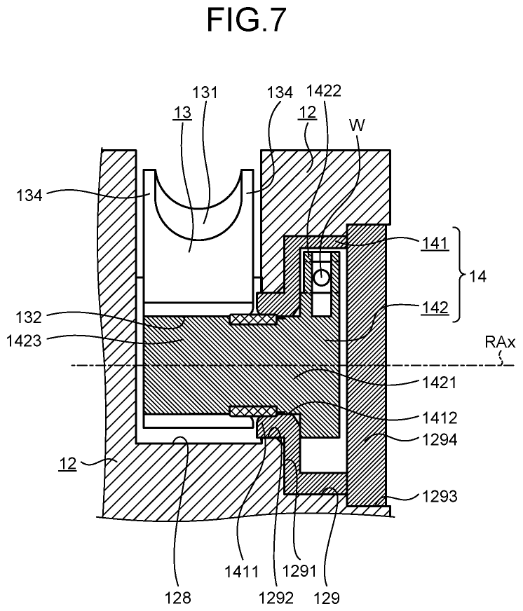

FIG. 7 is a diagram for description a second modified example of the embodiment. Specifically, FIG. 7 a diagram corresponding to FIG. 5.

In the above described embodiment, the hole 132 may be shaped like a hole 132 of the second modified example illustrated in FIG. 7.

Specifically, the hole 132 according to the second modified example is a through hole penetrating both side surfaces of the treatment tool raising stand 13, the side surfaces being along a rotation axis RAx, and similarly to the above described embodiment, the hole 132 has a first plane 1321 and a second plane 1322.

Even when the above described hole 132 according to the second modified example is adopted, effects similar to those of the embodiment described above are achieved.

An endoscope distal end and an endoscope, according to the disclosure, enable improvement in insertability of treatment tools.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A distal end portion for use with an endoscope, the distal end portion comprising:
a distal end body having an insertion hole, the insertion hole configured to accept a treatment tool inserted therethrough;
a shaft rotatably disposed relative to the distal end body; and
a raising base configured to rotate with the shaft, and be movable between a raised state where the raising base has been raised relative to a longitudinal direction of the distal end body and a laid state where the raising base has been laid relative to the longitudinal direction, and the raising base comprising;
a shaft hole having an interior configured to be engaged with an exterior of the shaft; and
a treatment tool guiding surface configured to abut against the treatment tool to adjust a direction in which the treatment tool protrudes, the treatment tool guiding surface provided on at least a proximal end side of the raising base, wherein
the interior of the shaft hole comprises a first surface configured to abut against a corresponding portion of the exterior of the shaft, the first surface is positioned at a shortest distance from the treatment tool guiding surface relative to other portions of the interior of the shaft hole, and
the treatment tool guiding surface is configured to intersect the longitudinal direction in the laid state.

2. The distal end portion according to claim 1, wherein when the raising base is in the laid state, the treatment tool guiding surface extends in a direction in which the insertion hole extends.

3. The distal end portion according to claim 1, wherein a cross section of the treatment tool guiding surface has a linear shape, the cross section being orthogonal to the longitudinal direction of the distal end body.

4. The distal end portion according to claim 3, wherein
the interior of the shaft hole further having a second surface configured to abut against an other corresponding surface of the exterior of the shaft, and
the first surface and the second surface are at an acute angle relative to each other.

5. The distal end portion according to claim 4, wherein the raising base further comprising a screw hole communicating with the shaft hole, and
the distal end portion further comprising a screw inserted into the screw hole for biasing the shaft against the first surface and the second surface.

6. The distal end portion according to claim 1, wherein the shaft hole is a through hole.

7. The distal end portion according to claim 1, wherein the shaft hole is a blind hole.

8. The distal end portion according to claim 1, wherein a cross section of the treatment tool guiding surface has a curved shape, the cross section being orthogonal to the longitudinal direction of the distal end body.

9. The distal end portion according to claim 8, wherein
the interior of the shaft hole further having a second surface configured to abut against an other corresponding portion of the exterior of the shaft, and
the first surface and the second surface are at an acute angle relative to each other.

10. The distal end portion according to claim 9, wherein the raising base further comprising a screw hole communicating with the shaft hole, and
the distal end portion further comprising a screw for biasing the shaft against the first surface and the second surface.

11. The distal end portion according to claim 8, wherein the raising base has a third surface on a periphery of the treatment tool guiding surface.

12. The distal end portion according to claim 1, further comprising an ultrasound probe positioned distally relative to the raising base.

13. An endoscope comprising:
an insertion portion having a distal end portion at a distal end of the insertion portion, the insertion portion being configured to be inserted into a subject, wherein
the distal end portion comprises:
a distal end body having an insertion hole, the insertion hole configured to accept a treatment tool inserted therethrough;
a shaft rotatably disposed relative to the distal end body; and
a raising base configured to rotate with the shaft, and be movable between a raised state where the raising base has been raised relative to a longitudinal direction of the distal end body and a laid state where the raising base has been laid relative to the longitudinal direction, and the raising base comprising;
a shaft hole having an interior configured to be engaged with an exterior of the shaft; and
a treatment tool guiding surface configured to abut against the treatment tool to adjust a direction in which the treatment tool protrudes, the treatment tool guiding surface provided on at least a proximal end side of the raising base, wherein
the interior of the shaft hole comprises a first surface configured to abut against a corresponding portion of the exterior of the shaft, the first surface is positioned at a shortest distance from the treatment tool guiding surface relative to other portions of the interior of the shaft hole, and
the treatment tool guiding surface is configured to intersect the longitudinal direction in the laid state.

14. The endoscope according to claim 13, wherein when the raising base is in the laid state, the treatment tool guiding surface extends in a direction in which the insertion hole extends.

15. The endoscope according to claim 13, wherein the distal end portion further comprises an ultrasound probe that is positioned distally relative to the raising base.
